(19) United States
(12) Patent Application Publication
(10) Pub. No.: US 2021/0236022 A1
(43) Pub. Date: Aug. 5, 2021

(54) Smart Interpretive Wheeled Walker using Sensors and Artificial Intelligence for Precision Assisted Mobility Medicine Improving the Quality of Life of the Mobility Impaired

(71) Applicant: ProtoStar, Inc., a Delaware Corporation, San Diego, CA (US)

(72) Inventors: Craig Timothy Shugert, Rancho Santa Fe, CA (US); Andrew Erwin Senyei, La Jolla, CA (US)

(73) Assignee: ProtoStar, Inc., a Delaware Corporation, San Diego, CA (US)

(21) Appl. No.: 17/165,554

(22) Filed: Feb. 2, 2021

Related U.S. Application Data
(60) Provisional application No. 62/970,111, filed on Feb. 4, 2020.

(51) Int. Cl. A61B 5/11; A61B 5/00; A61H 3/04; G16H 20/30; G16H 40/67; G16H 50/20; G06N 20/00

(52) U.S. Cl. CPC A61B 5/112; A61B 5/1128; A61B 5/6887; A61B 5/7264; A61B 5/1117; A61B 5/0022; A61H 2003/006; G16H 20/30; G16H 40/67; G16H 50/20; G06N 20/00; A61H 2201/5092; A61H 2201/5097; A61H 3/04

(57) ABSTRACT

A system includes a wheeled walker having a frame and a plurality of wheel assemblies coupled to the frame for supporting the frame above a walking surface, the frame and the plurality of wheel assemblies defining a volume above the walking surface occupied by at least a portion of a user's legs as the user walks on the walking surface using the wheeled walker, the wheeled walker further having a camera directed toward the volume. The system may include a non-transitory program storage medium storing instructions executable by a processor or programmable circuit to collect image data from the camera, evaluate the user's gait based on the image data, and output a result of the evaluation.

### CROSS-REFERENCE TO RELATED APPLICATIONS

The present application claims the benefit of U.S. Provisional Application No. 62/970,111, filed Feb. 4, 2020, the contents of which are expressly incorporated herein by reference.

### STATEMENT RE: FEDERALLY SPONSORED RESEARCH/DEVELOPMENT

Not Applicable

### BACKGROUND

### Technical Field

The present disclosure relates generally to assistive mobility devices and, more particularly, to a wheeled walker or rollator.

### Background

Assistive mobility devices, including wheeled walkers (also known as rollators), are widely used by mobility impaired individuals. A detailed discussion of the use and classification of assistive mobility devices is found in U.S. Pat. No. 9,585,807 to Fellingham, issued on Mar. 7, 2017 and entitled Collapsible Upright Wheeled Walker Apparatus (“Fellingham”), the entire disclosure of which is incorporated herein by reference.

Many wheeled walkers are not designed to support significant user weight during use and are used for the accepted purpose of providing assistance in balance and gait. Use of such devices requires the user to engage the wheeled walker with the hands and wrists alone, often with a stooping and leaning posture. Fellingham discloses an apparatus with raised adjustable forearm support elements to provide upper body support to a user, allowing the wheeled walker to support a significant amount of a user's weight while the user is walking. Fellingham discloses allowing a user to engage the wheeled walker in an upright walking position supported by the user's forearms while grasping two forward hand grips. The upright walking posture has the advantages of reducing heart and lung compression, improving circulation, and providing the therapeutic effects of longer walking times. Other patent documents that disclose wheeled walkers supporting an upright walking posture with supports for the user's upper body or forearms include U.S. Pat. No. 10,307,321 to Pan, issued on Jun. 4, 2019 and entitled Wheeled Walker with a Movable Seat (“Pan”) and U.S. Patent Application Pub. No. 2019/0105222 to Fellingham, filed Oct. 2, 2018 and entitled Wheeled Walker (“Fellingham II”), the entire disclosure of both Pan and Fellingham II being incorporated herein by reference.

With the aging demographic of 55 million people over the age of 65 and increasing number of patients with neurological disorders, there is an increasing need for improved mobility devices with sensing and monitoring technologies.

### BRIEF SUMMARY

The present disclosure contemplates various systems, methods, and apparatuses for overcoming the above drawbacks accompanying the related art. One aspect of the embodiments of the present disclosure is a wheeled walker including a frame and a plurality of wheel assemblies coupled to the frame for supporting the frame above a walking surface, the frame and the plurality of wheel assemblies defining a volume above the walking surface occupied by at least a portion of a user's legs as the user walks on the walking surface using the wheeled walker. The wheeled walker may further include a camera directed toward the volume and a wireless transmitter for wirelessly transmitting image data collected from the camera to a mobile device.

The wireless transmitter may wirelessly transmit the image data to the mobile device according to a wireless communication protocol having a range of approximately ten meters or less.

The wheeled walker may comprise a forearm gutter, a height adjustment tube movable relative to the frame, and an upper support joint connected to the forearm gutter and to the height adjustment tube. The camera may be mounted to the upper support joint underneath the forearm gutter.

Another aspect of the embodiments of the present disclosure is a system including a wheeled walker having a frame and a plurality of wheel assemblies coupled to the frame for supporting the frame above a walking surface, the frame and the plurality of wheel assemblies defining a volume above the walking surface occupied by at least a portion of a user's legs as the user walks on the walking surface using the wheeled walker, the wheeled walker further having a camera directed toward the volume. The system may further include a non-transitory program storage medium storing instructions executable by a processor or programmable circuit to collect image data from the camera, evaluate the user's gait based on the image data, and output a result of the evaluation.

The instructions may be executable to evaluate the user's gait further based upon contextual information associated with the user. The contextual information may include one or more items of information selected from the group consisting of a current diagnosis, a historical diagnosis, a medication, a medical test result, a score, and a health monitoring device measurement. The evaluating of the user's gait may include comparing the collected image data and contextual information to a machine learning corpus derived at least in part from image data and contextual information of different users.

The evaluating of the user's gait may include comparing the collected image data to a machine learning corpus derived at least in part from image data of different users.

The instructions may be executable to evaluate the user's gait further based upon past image data associated with the user's gait.

The result of the evaluation may comprise a medical diagnosis of the user.

The result of the evaluation may comprise a detection that the user has fallen.

The result of the evaluation may comprise feedback to the user.

The non-transitory program storage medium may be included in a mobile device including a processor or programmable circuit for executing the instructions. The evaluating of the user's gait may include wirelessly transmitting the image data to a server and receiving the result of the evaluation from the server. The server may be at least partly embodied in a cloud-based machine learning platform.

Another aspect of the embodiments of the present disclosure is a system including a wheeled walker having a frame and a plurality of wheel assemblies coupled to the frame for supporting the frame above a walking surface, the frame and the plurality of wheel assemblies defining a volume above the walking surface occupied by at least a portion of a user's legs as the user walks on the walking surface using the wheeled walker, the wheeled walker further having a camera directed toward the volume. The system may further include a non-transitory program storage medium storing instructions executable by a processor or programmable circuit to collect image data from the camera, send the collected image data to a remote server for processing using artificial intelligence, and receive an evaluation of the user's gait from the remote server based upon the collected image data.

The evaluation may be further based upon contextual information associated with the user. The contextual information may include one or more items of information selected from the group consisting of a current diagnosis, a historical diagnosis, a medication, a medical test result, a score, and a health monitoring device measurement. The processing using artificial intelligence may include comparing the collected image data and contextual information to a machine learning corpus derived at least in part from image data and contextual information of different users.

The processing using artificial intelligence may include comparing the collected image data to a machine learning corpus derived at least in part from image data of different users.

The evaluation may be further based upon past image data associated with the user's gait.

The evaluation may comprise a detection that the user has fallen.

The remote server may be at least partly embodied in a cloud-based machine learning platform.

Another aspect of the embodiments of the present disclosure is a method of evaluating a gait of a user of a wheeled walker. The method may include collecting image data of the user's legs and/or feet as the user walks using the wheeled walker from a camera disposed on the wheeled walker, sending the collected image data to a remote server for processing using artificial intelligence, and receiving an evaluation of the user's gait from the remote server based upon the collected image data.

The evaluation may be further based upon contextual information associated with the user. The contextual information may include one or more items of information selected from the group consisting of a current diagnosis, a historical diagnosis, a medication, a medical test result, a score, and a health monitoring device measurement. The processing using artificial intelligence may include comparing the collected image data and contextual information to a machine learning corpus derived at least in part from image data and contextual information of different users.

The processing using artificial intelligence may include comparing the collected image data to a machine learning corpus derived at least in part from image data of different users.

The evaluation may be further based upon past image data associated with the user's gait.

The evaluation may comprise a detection that the user has fallen.

The remote server may be at least partly embodied in a cloud-based machine learning platform.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 shows a system according to an embodiment of the present disclosure, including a wheeled walker, a mobile device, and a remote server;

FIG. 2 is a front view of the wheeled walker and mobile device;

FIG. 3 is a cross-sectional view of the wheeled walker and mobile device taken along the line 3-3 in FIG. 2;

FIG. 4 is a closeup view of the wheeled walker and mobile device, showing a camera beneath a forearm gutter of the wheeled walker;

FIG. 5 is a front view of the camera;

FIG. 6 is a bottom view of the camera taken along the line 6-6 in FIG. 5;

FIG. 7 is a side view of the camera;

FIG. 8 is a schematic view of a gait evaluation apparatus according to an embodiment of the present disclosure; and

FIG. 9 shows an example operational flow according to an embodiment of the present disclosure.

### DETAILED DESCRIPTION

The present disclosure encompasses various embodiments of systems, methods, and apparatuses for observing and diagnosing a user's gait while using a wheeled walker. The detailed description set forth below in connection with the appended drawings is intended as a description of several currently contemplated embodiments and is not intended to represent the only form in which the disclosed device may be developed or utilized. The description sets forth the functions and features in connection with the illustrated embodiments. It is to be understood, however, that the same or equivalent functions may be accomplished by different embodiments that are also intended to be encompassed within the scope of the present disclosure. It is further understood that the use of relational terms such as first and second and the like are used solely to distinguish one from another entity without necessarily requiring or implying any actual such relationship or order between such entities.

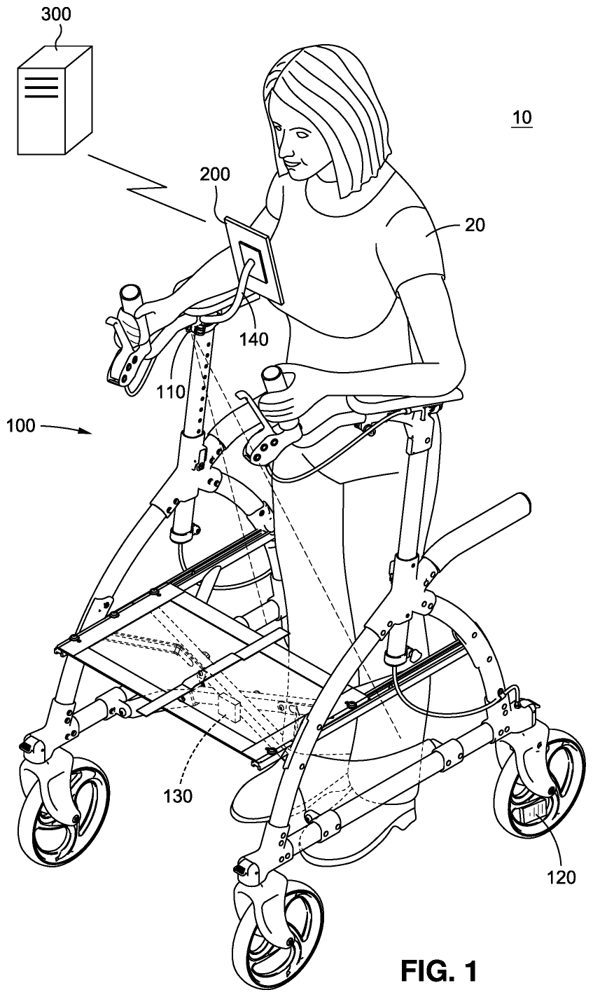

FIG. 1 shows a system 10 according to an embodiment of the present disclosure, including a wheeled walker 100, a mobile device 200 (e.g. a smartphone or tablet), and a remote server 300. The wheeled walker 100 may include one or more cameras 110 directed toward the legs and/or feet of a user 20 of the wheeled walker 100. As the user 20 walks along using the wheeled walker 100, the one or more cameras 110 may capture images of the user's gait. A wireless transmitter 112 (see FIG. 5) may wirelessly transmit image data collected from the one or more cameras 110 to the mobile device 200, which may be docked with the wheeled walker 100 for easy access by the user 20 as shown. A mobile application running on the mobile device 200 may then cause a processor of the mobile device 200, alone or together with a processor of a remote server 300, to evaluate the user's gait based on the collected image data. The evaluation may further be based on data collected from various other sensors that may be included on the wheeled walker 100, for example, motion sensors 120 such as gyroscopes and/or accelerometers disposed on the wheels, proximity sensors 130 such as cameras or ultrasonic, LED, or laser (e.g. VCSEL) detectors disposed on the front of the wheeled walker 100, additional cameras facing the user's head and/or upper body, health monitoring devices (e.g. a pulse-oximeter), etc. The mobile device 200 may then output the result of the evaluation to the user 20 in the form of a warning, recommendation, or other feedback, while the mobile device 200 and/or remote server 300 may additionally notify a third party such as the user's primary care physician.

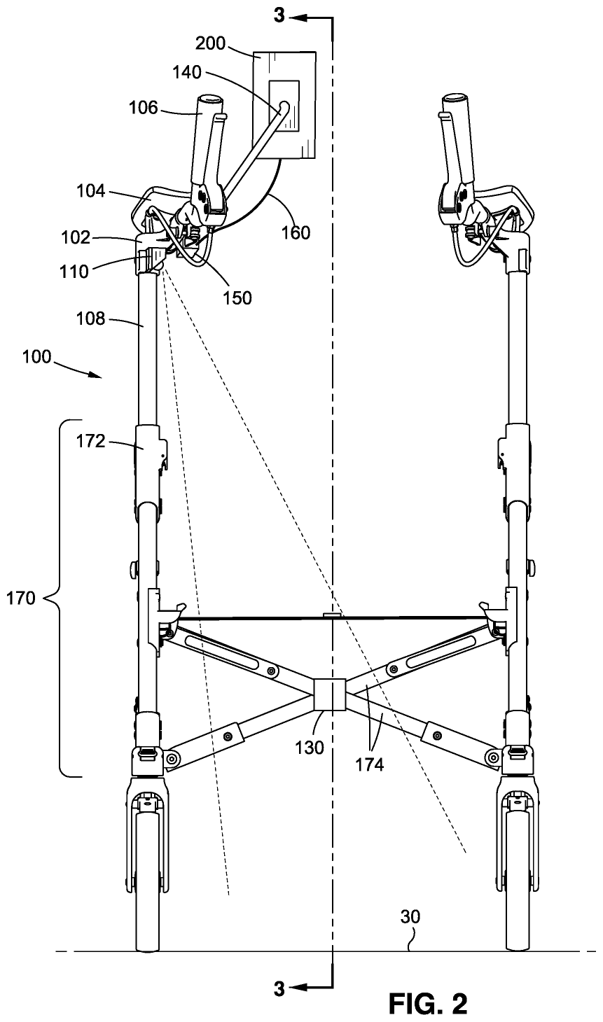

FIG. 2 is a front view of the wheeled walker 100 and mobile device 200. In the case of a wheeled walker 100 that provides upper body support as in the illustrated example (which may be a rollator as disclosed by Fellingham, Pan, or Fellingham II, for example), each of the one or more cameras 110 may be mounted to an upper support joint 102 underneath a forearm gutter 104 of the wheeled walker 100. The one or more cameras 110 may thus face downward toward the user's legs and/or feet. There may be a single camera 110 as shown or there may be a plurality of cameras 110, e.g., one provided underneath each forearm gutter 104. Providing each camera 110 on the upper support joint 102 rather than the forearm gutter 104 itself allows for an unobtrusive placement of the camera 110 while still maintaining a clear view of the user's legs and/or feet. As such, the camera 110 is not likely to be accidentally bumped as the user 20 rests her forearm in the forearm gutters 104 and grips upper handles 106. In addition to being connected to a respective forearm gutter 104 and upper handle 106, each upper support joint 102 may be connected to a respective height adjustment tube 108. The height of each forearm gutter 104 may be adjustable by moving the respective height adjustment tube 108 up and down relative to a frame 170 of the wheeled walker 100 (or, more specifically, a respective frame top joint 172 of the frame 170). By providing each camera 110 on the upper support joint 102 rather than the height adjustment tube 108 itself, the camera 110 can be prevented from interfering with the full range of height adjustment as the height adjustment tube 108 approaches the frame top joint 172.

A mount 140 for the mobile device 200 may also be provided on the upper support joint 102, typically on only a single side of the wheeled walker 100. The mount 140 may comprise, for example, a jointed or flexible arm terminating in a mounting surface on which the mobile device 200 may be docked. As shown, the mount may extend inward from the upper support joint 102 toward the interior of the wheeled walker 100 rather than jutting outward. In this way, a potentially hazardous extension beyond the footprint of the wheeled walker 100 can be avoided. The mount 140 may position the mobile device 200 at a comfortable viewing distance from the user 20 so that the user 20 may view a screen of the mobile device 200 as needed (e.g. to receive feedback from a mobile application as described herein) while walking or standing using the wheeled walker 100.

A battery pack 150 (e.g. a rechargeable lithium-ion or lithium-polymer based power bank) for powering the one or more cameras 110, mobile device 200, and/or other sensors 130, 140 may also be provided on the upper support joint 102, for example, behind the camera 110. For ease of illustration, a single wire 160 (e.g. a USB cable) is shown connecting the mobile device 200 to the battery pack 150. However, it is contemplated that additional wires may be provided along the exterior of the height adjustment tubes 108 and frame 170 of the wheeled walker 100, and/or provided internally thereto, in order to provide battery power to the one or more cameras 110 and/or other sensors 120, 130 of the wheeled walker 100. Alternatively, one or more of the cameras 110 and/or sensors 120, 130 may include its own power source to reduce the amount of wires.

One or more proximity sensors 130 may be disposed on the frame 170 of the wheeled walker 100 facing outward to detect the proximity of the wheeled walker 100 to obstacles and/or designated landmarks or checkpoints. A proximity sensor 130 may be disposed in the center of the frame 170 on the front of the wheeled walker 100, for example, on an anterior bar of an X-folder system 174 as shown. However, other positions on the frame 170 are contemplated as well, including on the sides and rear of the wheeled walker 100. The proximity sensor(s) 130 may be cameras, ultrasonic proximity sensors, microwave Doppler sensors, or 3D infrared (IR) sensors, for example. In addition to generating data for the evaluation of the user's gait as described in more detail below, the proximity sensor(s) 130 may be used as part of a landmark-based location-tracking system or as part of an obstacle avoidance system as disclosed by U.S. Patent Application Pub. No. 2017/0258664 to Purcell, filed on Jan. 26, 2017 and entitled Upright Walker Having a User Safety System Employing Haptic Feedback (“Purcell”), the entire disclosure of which is incorporated herein by reference.

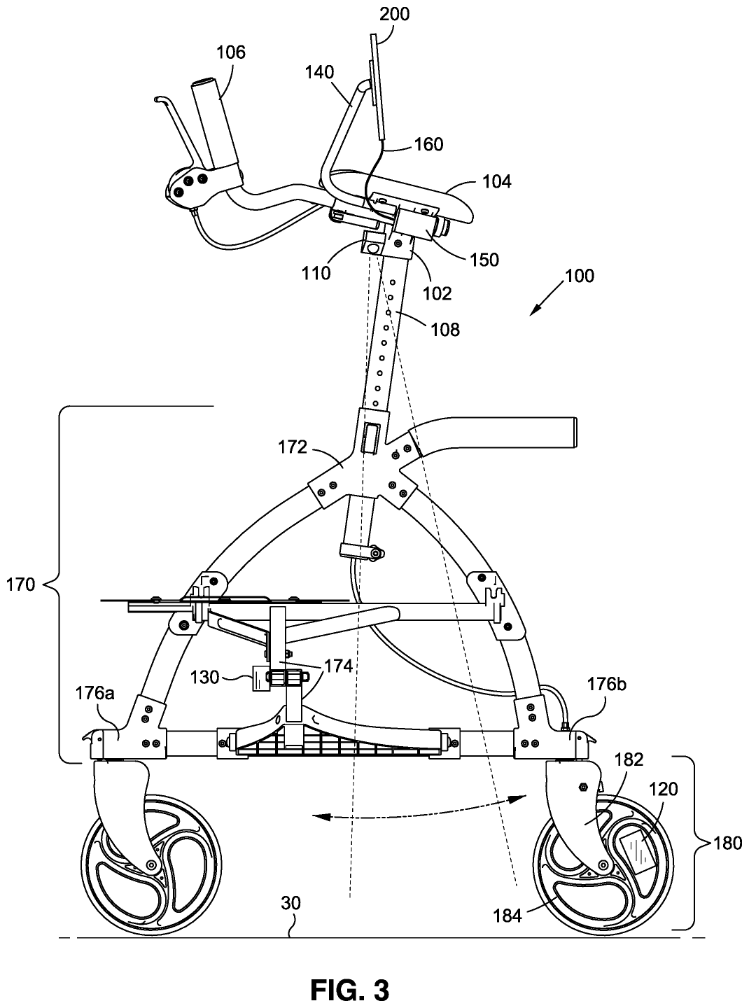

FIG. 3 is a cross-sectional view of the wheeled walker 100 and mobile device 200 taken along the line 3-3 in FIG. 2. As shown, the frame 170 of the wheeled walker 100 may be supported on a walking surface 30 by a plurality of wheel assemblies 180, each including a wheel fork 182 and a wheel 184. In this regard, the frame 170 may include, in addition to the frame top joints 172 and X-folder system 174 referred to above, a plurality of frame bottom joints 176a, 176b by which the frame 170 is connected to and supported by the wheel assemblies 180. As illustrated, for example, the plurality of frame bottom joints 176 may include left and right frame front joints 176a as well as left and right frame rear joints 176b, each connected to a wheel fork 182 of a respective wheel assembly 180.

One or more motion sensors 120 may be disposed on respective wheels 184 of the wheeled walker 100. The motion sensors 120 may comprise accelerometers and/or gyroscopes, which may be combined in various ways to produce “soft” sensor output, such as a pedometer reading of the user 20, a speed, acceleration, or direction of the user 20, and/or a tilt or attitude of the wheeled walker 100 (which may indicate a fall or a dangerous inclination that could potentially lead to a fall). Such “soft” sensor output may be produced by a processor of the mobile device 200 running a mobile application as described herein.

As noted above in relation to FIG. 1, the one or more cameras 110 may be directed toward the legs and/or feet of the user 20 of the wheeled walker 100. More generally, with reference to FIGS. 1-3, the frame 170 and the plurality of wheel assemblies 180 may define a volume or space above the walking surface 30 that is typically occupied by at least a portion of the user's legs as the user 20 walks on the walking surface 30 using the wheeled walker 100. The volume may be, for example, a rectangular prism extending upward from a square on the walking surface 30 whose corners are the four wheel assemblies 180 to the height of the frame top joints 172 or higher (e.g. to the height of one or both of the forearm gutters 104). In the case of a wheeled walker 100 having three wheels, the volume may be a triangular prism similarly defined. Generally speaking, the volume may be a geometric prism of arbitrary height extending upward from the footprint of the wheeled walker 100. Each of the one or more cameras 110 may be directed toward the volume (e.g. having at least a portion of the volume within its field of view). In this way, the one or more cameras 110 may be directed toward the user's legs and/or feet while the wheeled walker 100 is in use.

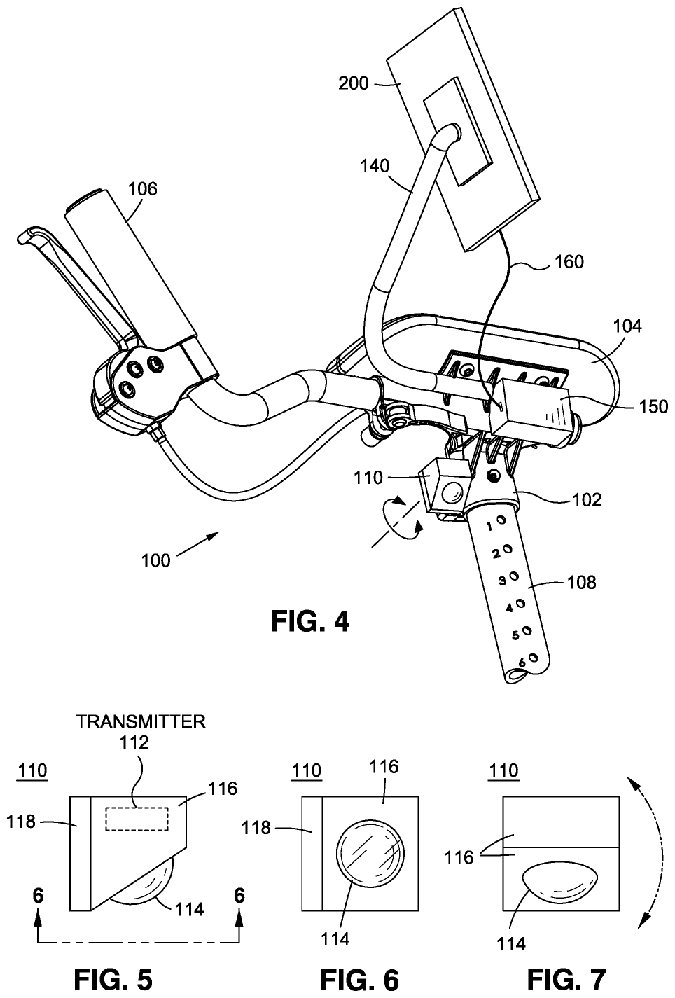

FIG. 4 is a closeup view of the wheeled walker 100 and mobile device 200, showing the camera 110 beneath the forearm gutter 104 of the wheeled walker 100. FIGS. 5-7 are closeup views of the camera 110, with FIG. 5 being a front view, FIG. 6 being a bottom view taken along the line 6-6 in FIG. 5, and FIG. 7 being a side view. The camera 110 may have wireless communication functionality and may be, for example, a Bluetooth-enabled camera. To this end, the camera 110 may include a wireless transmitter 112 as shown in FIG. 5, in addition to a lens 114, image sensor, etc. The wireless transmitter 112 may wirelessly transmit image data collected from the camera 110 to the mobile device 200. The image data may be transmitted according to a short-range wireless communication protocol such as Bluetooth. For example, the wireless communication protocol may have a range of approximately ten meters or less. In this way, the wireless transmitter 112 may be used to transmit the image data to the mobile device 200 while the mobile device 200 is docked with the wheeled walker (e.g. via the mount 140). In the case of multiple cameras 110 and/or additional sensors 120, 130 as described above, the wireless transmitter 112 may be shared by the plurality of cameras 110 and sensors 120, 130. To this end, the various devices may be connected by wires as described above in relation to the battery pack 150. Alternatively, some or all of the additional devices may have their own respective wireless transmitters 112.

The housing of the camera 110 may include a movable part 116 and a stationary part 118 (e.g. a mounting bracket), with the lens 114 provided on the movable part 116. In this way, the camera 110 may be rotatable or otherwise movable, with the movable part 116 rotating and/or translating relative to the stationary part 118. An example rotation of the camera 110 may be best seen in FIGS. 4 and 7, where it is depicted that the camera 110 may rotate so as to point more forward or more rearward relative to the wheeled walker 100. This axis of rotation may also be understood from the arrows in FIG. 3. By allowing the camera 110 to be rotated forward and rearward in this way, the camera 110 may be adjusted to properly aim at the user's legs and/or feet depending on the typical posture and walking style of the user 20. For example, the gait of a user 20 who stands upright may be more readably observed by a forward-angled camera 110, while the gait 20 of a user who hunches over and leans on the wheeled walker 100 with their legs further behind them may be more readably observed by a rearward-angled camera 110. It is contemplated that other axes of rotation may alternatively or additionally be provided, including 360-degree rotation.

In FIG. 5, the wireless transmitter 112 is illustrated as being provided in the movable part 116 of the camera 110. However, the wireless transmitter 112 may instead be provided in the stationary part 118 or may be provided as a separate device entirely outside the housing of the camera 110. It should also be noted that the term “transmitter,” as used herein is not intended to be limited to devices with exclusive transmission functionality and may also refer to transceivers.

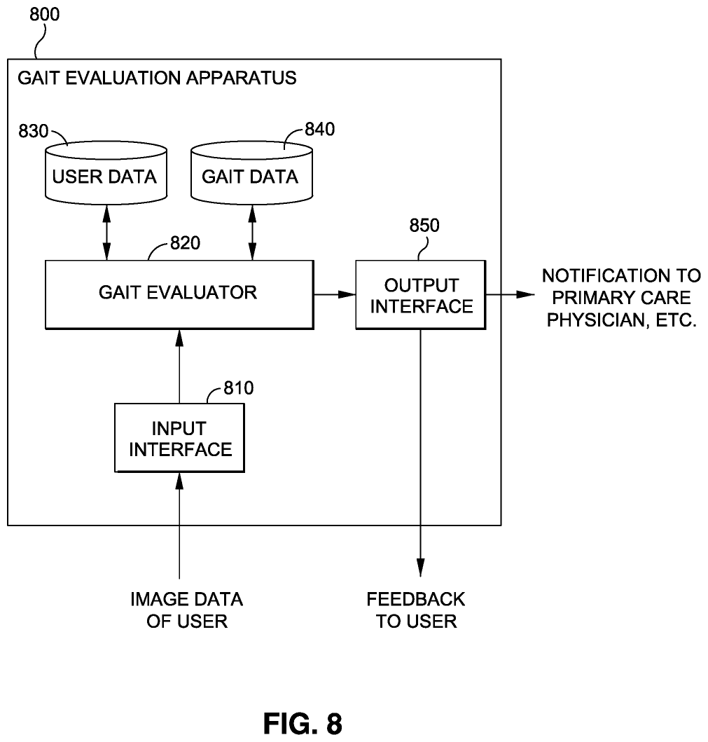

FIG. 8 is a schematic view of a gait evaluation apparatus 800 according to an embodiment of the present disclosure. The gait evaluation apparatus 800 may be a server or a combination of networked servers (e.g. a cloud) that interacts with a mobile application installed on the mobile device 200 in order to evaluate the gait of the user of the wheeled walker 100 based on image data collected from the one or more cameras 110 and/or other additional sensors 120, 130 (which may themselves comprise additional cameras as mentioned above). In this regard, the gait evaluation apparatus 800 may be embodied in the remote server 300 depicted in FIG. 1. Alternatively, all or a portion of the gait evaluation apparatus 800 may be embodied in the mobile application installed on the mobile device 200. The gait evaluation apparatus 800 may include an input interface 810, a gait evaluator 820, a user data storage 830, a gait data storage 840, and an output interface 850.

The input interface 810 may receive image data and other sensor data collected from the one or more cameras 110 and/or additional sensors 120, 130. In a case where the gait evaluation apparatus 800 is embodied in the remote server 300, the data may be received from the mobile device 200 over a network such as the Internet. To this end, the mobile device 200 may include a radio frequency transceiver with WiFi and/or cellular communication functionality in addition to the short-range (e.g. Bluetooth) communication functionality used by the mobile device 200 to interface with the cameras 110 and/or additional sensors 120, 130 of the wheeled walker 100. In accordance with software instructions embodied in the mobile application installed on the mobile device 200, a processor of the mobile device 200 may pre-process the data received from the wheeled walker 100 and send the data to the remote server 300 to be received by the input interface 810. The preprocessing of the data may include, for example, filtering the data according to calibration settings of the cameras 110 and sensors 120, 130 (which may be adjustable in the mobile application), generating “soft” sensor data such as pedometer data using a combination of sensors (e.g. accelerometers and gyroscopes of one or more motion sensors 120), and packaging the data according to a preferred transmission schedule (e.g. real-time transmission for data that is relevant to user feedback and emergency notifications, daily transmission for data that is only relevant to recordkeeping, etc.). The preprocessing of the data may further include appending user identifying information identifying the particular user 20, which may be determined according to a user login credential stored by the mobile application and/or facial recognition based on image data collected from a user-facing camera of the wheeled walker 100, appending device identifying information identifying the particular model of the wheeled walker 100 as specified per application settings, appending time-stamped location data (e.g. GPS data) collected by the mobile device 200, etc.

The gait evaluator 820 may evaluate the user's gait based on the image data and/or other data received by the input interface 810. For example, the gait evaluator 820 may compare the received image data (e.g. video or still images) and/or other sensor data to user data stored in the user data storage 830 and/or gait data stored in the gait data storage 840. The user data stored in the user data storage 830 may represent information indexed by the particular user of the wheeled walker 100, which may include, for example, medical records including current and historical diagnoses, medications, medical test results, scores, health monitoring device measurements, etc., as well as past image data and/or other sensor data associated with the user's gait. The gait evaluator 820 may compare the received data to the relevant user data of the particular user 20 of the wheeled walker 100 by referring to user identifying information associated with the received data, which may be appended to the data by the mobile device 200 as described above. In a case where the gait evaluator 820 and user data storage 830 are included on the mobile device 100 itself rather than a remote server 300, it is contemplated that the user data storage 830 may in some cases contain only a single user's data, such that it may not be necessary for the gait evaluator 820 to identify the current user of the wheeled walker 100. By referring to the user data storage 830, the gait evaluator 820 may discern any gait behavior that is out of the ordinary for the particular user, with past behavior of the user being used as a baseline for purposes of comparison.

The information stored in the user data storage 830 may further supplement the received image data and/or other sensor data with relevant contextual information, such as a current diagnosis or medication of the user 20. For example, a user 20 who is currently taking Levodopa for Parkinson's disease may be expected to experience side effects that affect the user's gait as the medication wears off, causing the user 20 to shuffle her feet or exhibit a freeze in her gait. The gait evaluator 820 may combine such contextual information from the user data storage 830 with the received image data and/or other sensor data (possibly including additional medical sensor data such as current pulse-oximeter measurements or other vital signs and/or external motion sensor data such as from an accelerometer and/or gyroscope in a smartwatch or pendant worn by the user 20) to conduct an artificial intelligence (AI) analysis of the user's gait. The AI analysis may, for example, interpret the way the user 20 is walking (e.g. movement of feet, knees, legs, and hips) and compare the user's gait to past data of the user and/or other known data collected from previously diagnosed patients to suggest that a health condition might exist, detect a fall, or provide feedback on ways to improve the user's gait. To this end, the gait data stored in the gait data storage 840 may represent a machine learning corpus derived from image data and/or other sensor data, medical records, etc. of many different users. In this regard, the gait evaluator 820 and/or gait data storage 840 may interact with and/or be wholly or partly embodied in a cloud-based machine learning platform, such as Microsoft Azure Machine Learning, IBM Watson, Google Cloud AI, or Amazon Machine Learning.

The output interface 850 may output a result of the evaluation by the gait evaluator 820. To continue with the example of the user 20 who is currently taking Levodopa for Parkinson's disease, the gait evaluator 820 may evaluate the user's gait as exhibiting shuffling consistent with a wearing off of Levodopa in the user 20. The output interface 850 may provide relevant feedback to the user, for example, causing the mobile device 200 to output (visually on a display or audibly from a speaker of the mobile device 200) a suggestion or recommendation to the user 20, e.g., “I have noticed you are shuffling your feet. Have you taken your prescribed medication, Levodopa? If you have, might I suggest you sit down and take a break? Once rested, remember to lift your feet while walking.” Such feedback may serve as a gait training tool for the user 20. For example, the feedback may include real-time instructions and reminders regarding the user's gait (e.g. “lift left foot-lift right foot-lift left foot” etc.) and may tell the user whether she is correctly lifting her feet to improve her gait and eliminate shuffling. The feedback may further include recommended exercise routines for improving the user's gait, with the mobile application on the mobile device 200 possibly allowing the user 20 to set personal goals and track progress.

As noted above, the proximity sensor(s) 130 may be used as part of a landmark-based location-tracking system or an obstacle avoidance system as disclosed by Purcell. In this respect, it is contemplated that the feedback provided to the user by the gait evaluation apparatus 800 may include verbal or other assistance to guide the user 20 around obstacles and/or to provide directions to the user 20 within a given setting such as a house or senior living center. By incorporating proximity data from a proximity sensor 130 and/or GPS data (e.g. from the mobile device 200 or a GPS receiver on the wheeled walker 100) into the AI analysis, the gait evaluation apparatus 800 may learn the typical surroundings of the user 20 and provide improved feedback accordingly. For example, the gait evaluation apparatus 800 may learn the floorplan of the user's house and the typical movements of the user 20 within the floorplan.

In addition to outputting feedback to the user 20 as the result of the evaluation, the output interface 850 may further output a resulting diagnosis or other notification to a third-party device associated with a primary care physician, family member, record keeper, or other interested party. For example, in the case of the user 20 who recently received the feedback regarding taking Levodopa and/or a break, the gait evaluation apparatus 800 may continue to monitor the user's gait to see if the user 20 has corrected her gait. If the symptoms persist (e.g. for a predetermined period of time or after a predetermined escalation of increasingly drastic feedback to the user), the output interface 850 may send a notification to a mobile device of the user's caretaker. Upon receiving the notification, the caretaker may check on the user 20 to assist the user 20 and perhaps prevent a fall. It is also contemplated that the notification may include the image data of the user's gait, such as a video stream, to be provided to a medical professional for human evaluation and treatment of medical conditions.

In some cases, such a notification may occur simultaneously with the feedback to the user 20, without delay, as in the case of a gait evaluation that requires urgent attention (e.g. gait data indicative of a fall or a loss of consciousness). Fall detection is especially important as one ages. One in three over the age of 65 will have a reportable fall each year. In some instances, one may be incapacitated for an extended period of time, unable to get to a phone to call for assistance. If the gait evaluator 820 detects a fall (e.g. based on the motion sensor data collected from the one or more motion sensors 120), the feedback provided to the user may include an interactive offer for assistance. For example, the mobile device 200 may say, “Fall detected. Please push green button on phone acknowledging you are okay or push red button to seek assistance.” The user's input to the mobile device 200, or the user's failure to make any input within a predetermined period of time, may initiate a notification to the user's caregiver.

As noted above, the motion sensor(s) 120 may be used to produce a pedometer or other distance reading, speed, acceleration, or direction of the user 20. Close to 5 million people in the United States have a memory disorder. Family members have increased concerns regarding these people wandering away and being reported lost. Early detection of someone walking away from their home or secure community using their wheeled walker is of vital concern. Based on sensor data from the motion sensor(s) 120 and/or GPS data, the gait evaluator 820 may detect that the user 20 has gone beyond a predefined set of coordinates (e.g. a geo-fence) representing a safe zone boundary defined by the user's family member or other caretaker. In such a case, the output interface 850 may notify the caretaker accordingly. Along the same lines, the gait evaluation apparatus 800 may provide a notification when the user 20 moves within her typical surroundings at a slower than normal speed or in a way that is otherwise out of the ordinary. For example, in the example of the evaluation apparatus 800 that has learned the floorplan of the user 20, the apparatus 800 may alert a caretaker when the user 20 has spent an unusually long time in the bathroom, which might indicate a fall or loss of consciousness.

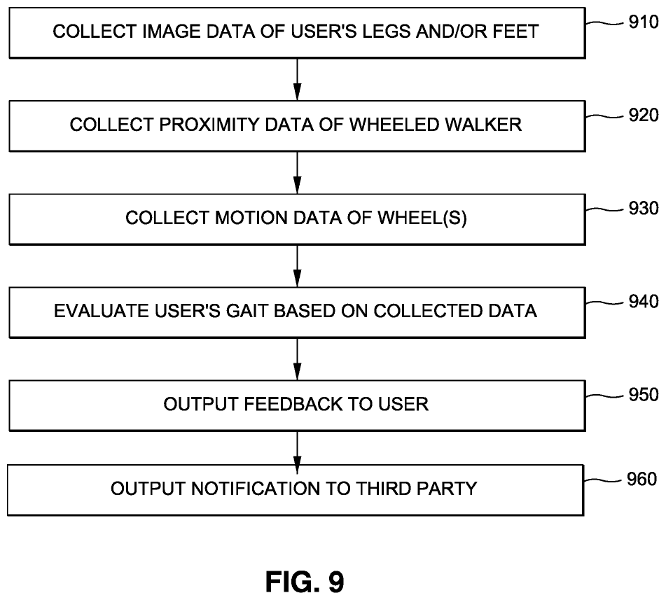

FIG. 9 shows an example operational flow according to an embodiment of the present disclosure. The operational flow shown in FIG. 9 may be performed entirely by the mobile device 200, entirely by a remote server 300, or by a combination of the mobile device 200 and the remote server 300. The operational flow may begin with the collection of image data of the user's legs and/or feet by one or more cameras 110 (step 910), the collection of proximity data of the wheeled walker 100 by one or more proximity sensors 130 (step 920), and/or the collection of motion data of the wheels 184 of the wheeled walker 100 by one or more motion sensors 120 (step 930). In the context of the mobile device 200, the collection of the image data and/or other sensor data of the user 20 may refer to the receipt of such data by short-range wireless communication (e.g. Bluetooth) from one or more wireless transmitters 116 on the wheeled walker 100. In the context of the remote server 300, the collection of the image data and/or other sensor data of the user 20 may refer to the receipt of pre-processed (or raw) data from the mobile device 200 over a network such as the Internet. In either case, such functionality may be regarded as performed by the input interface 810 of the gait evaluation apparatus 800 described above.

The operational flow may continue with the evaluation of the user's gait based on the collected data (step 940), which may be performed by a gait evaluator 820 as embodied in one or more processors located in the mobile device 200, the remote server 300, and/or a cloud-based machine learning platform. Lastly, the operational flow may conclude with the outputting of feedback to the user (step 950) and/or a notification to a third party (step 960). In the context of the mobile device 200, the output of feedback may refer to the generation by the mobile application of a display output, speaker output, haptic output, etc. of the mobile device 200 according to the result of the evaluation, while the output of the third-party notification may refer to the transmission of a signal to a third-party device over a network (e.g. the Internet) by the mobile device 200. In the context of the remote server 300, the output of the feedback or notification may instead refer to the transmission of a signal over a network (e.g. the Internet) to the mobile device 200 containing data for generating such feedback or notification, though the output of the third-party notification may be performed directly from the remote server 300.

In the above examples, it is generally assumed that a mobile device 200 is used in conjunction with the wheeled walker 100 to collect the camera and sensor data and provide feedback to the user 20, as well as in some cases to perform the gait evaluation locally. However, the disclosure is not intended to be so limited. For example, the wheeled walker 100 may have an onboard digital processor that performs some or all of the functions described in association with the mobile device 200, possibly including a radio frequency transceiver with WiFi and/or cellular communication functionality for communicating with a remote server 300, a cloud-based machine learning platform, and/or interested third parties. In the case of such an onboard digital processor, the mobile device 200 may not be needed and can be omitted. An onboard device of this type may be located, for example, on the upper support joint 102 of the wheeled walker 100 near the camera 110 and/or battery 150 (or may include the camera 110 and/or battery 150 within the same housing).

The functionality described in relation to the gait evaluation apparatus 800 of FIG. 8 and operational flow of FIG. 9, which may reside in a mobile device 200 and/or remote server 300 as shown in FIG. 1 (or alternatively in an onboard device as described above), may be wholly or partly embodied in one or more computers including a processor (e.g. a CPU), a system memory (e.g. RAM), and a hard drive or other secondary storage device. The processor may execute one or more computer programs (e.g. the mobile application described above), which may be tangibly embodied along with an operating system in a computer-readable medium, e.g., the secondary storage device. The operating system and computer programs may be loaded from the secondary storage device into the system memory to be executed by the processor. The computer may further include a network interface for network communication between the computer and external devices (e.g. over the Internet).

The computer programs may comprise program instructions which, when executed by the processor, cause the processor to perform operations in accordance with the various embodiments of the present disclosure. The computer programs may be provided to the secondary storage by or otherwise reside on an external computer-readable medium such as a DVD-ROM, an optical recording medium such as a CD or Blu-ray Disk, a magneto-optic recording medium such as an MO, a semiconductor memory such as an IC card, a tape medium, a mechanically encoded medium such as a punch card, etc. Other examples of computer-readable media that may store programs in relation to the disclosed embodiments include a RAM or hard disk in a server system connected to a communication network such as a dedicated network or the Internet, with the program being provided to the computer via the network. Such program storage media may, in some embodiments, be non-transitory, thus excluding transitory signals per se, such as radio waves or other electromagnetic waves. Examples of program instructions stored on a computer-readable medium may include, in addition to code executable by a processor, state information for execution by programmable circuitry such as a field-programmable gate arrays (FPGA) or programmable logic array (PLA).

The above description is given by way of example, and not limitation. Given the above disclosure, one skilled in the art could devise variations that are within the scope and spirit of the disclosed device disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A wheeled walker comprising:
a frame;
a plurality of wheel assemblies coupled to the frame for supporting the frame above a walking surface, the frame and the plurality of wheel assemblies defining a volume above the walking surface occupied by at least a portion of a user's legs as the user walks on the walking surface using the wheeled walker;
a camera directed toward the volume; and
a wireless transmitter for wirelessly transmitting image data collected from the camera to a mobile device.

2. The wheeled walker of claim 1, wherein the wireless transmitter wirelessly transmits the image data to the mobile device according to a wireless communication protocol having a range of approximately ten meters or less.

3. The wheeled walker of claim 1, further comprising:
a forearm gutter;
a height adjustment tube movable relative to the frame; and
an upper support joint connected to the forearm gutter and to the height adjustment tube.

4. The wheeled walker of claim 3, wherein the camera is mounted to the upper support joint underneath the forearm gutter.

5. A system comprising:
a wheeled walker having a frame and a plurality of wheel assemblies coupled to the frame for supporting the frame above a walking surface, the frame and the plurality of wheel assemblies defining a volume above the walking surface occupied by at least a portion of a user's legs as the user walks on the walking surface using the wheeled walker, the wheeled walker further having a camera directed toward the volume; and
a non-transitory program storage medium storing instructions executable by a processor or programmable circuit to collect image data from the camera, evaluate the user's gait based on the image data, and output a result of the evaluation.

6. The system of claim 5, wherein the instructions are executable to evaluate the user's gait further based upon contextual information associated with the user, the contextual information including one or more items of information selected from the group consisting of a current diagnosis, a historical diagnosis, a medication, a medical test result, a score, and a health monitoring device measurement.

7. The system of claim 6, wherein said evaluating the user's gait includes comparing the collected image data and contextual information to a machine learning corpus derived at least in part from image data and contextual information of different users.

8. The system of claim 5, wherein said evaluating the user's gait includes comparing the collected image data to a machine learning corpus derived at least in part from image data of different users.

9. The system of claim 5, wherein the instructions are executable to evaluate the user's gait further based upon past image data associated with the user's gait.

10. The system of claim 5, wherein the result of the evaluation comprises a medical diagnosis of the user.

11. The system of claim 5, wherein the result of the evaluation comprises a detection that the user has fallen.

12. The system of claim 5, wherein the result of the evaluation comprises feedback to the user.

13. The system of claim 5, wherein the non-transitory program storage medium is included in a mobile device including a processor or programmable circuit for executing the instructions.

14. The system of claim 13, wherein said evaluating the user's gait includes wirelessly transmitting the image data to a server and receiving the result of the evaluation from the server.

15. The system of claim 14, wherein the server is at least partly embodied in a cloud-based machine learning platform.

16. A system comprising:
a wheeled walker having a frame and a plurality of wheel assemblies coupled to the frame for supporting the frame above a walking surface, the frame and the plurality of wheel assemblies defining a volume above the walking surface occupied by at least a portion of a user's legs as the user walks on the walking surface using the wheeled walker, the wheeled walker further having a camera directed toward the volume; and
a non-transitory program storage medium storing instructions executable by a processor or programmable circuit to collect image data from the camera, send the collected image data to a remote server for processing using artificial intelligence, and receive an evaluation of the user's gait from the remote server based upon the collected image data.

17. The system of claim 16, wherein the evaluation is further based upon contextual information associated with the user, the contextual information including one or more items of information selected from the group consisting of a current diagnosis, a historical diagnosis, a medication, a medical test result, a score, and a health monitoring device measurement.

18. The system of claim 17, wherein said processing using artificial intelligence includes comparing the collected image data and contextual information to a machine learning corpus derived at least in part from image data and contextual information of different users.

19. The system of claim 16, wherein said processing using artificial intelligence includes comparing the collected image data to a machine learning corpus derived at least in part from image data of different users.

20. The system of claim 16, wherein the evaluation is further based upon past image data associated with the user's gait.

21. The system of claim 16, wherein the evaluation comprises a detection that the user has fallen.

22. The system of claim 16, wherein the remote server is at least partly embodied in a cloud-based machine learning platform.

23. A method of evaluating a gait of a user of a wheeled walker, the method comprising:
collecting image data of the user's legs and/or feet as the user walks using the wheeled walker from a camera disposed on the wheeled walker;
sending the collected image data to a remote server for processing using artificial intelligence; and
receiving an evaluation of the user's gait from the remote server based upon the collected image data.

24. The method of claim 23, wherein the evaluation is further based upon contextual information associated with the user, the contextual information including one or more items of information selected from the group consisting of a current diagnosis, a historical diagnosis, a medication, a medical test result, a score, and a health monitoring device measurement.

25. The method of claim 24, wherein said processing using artificial intelligence includes comparing the collected image data and contextual information to a machine learning corpus derived at least in part from image data and contextual information of different users.

26. The method of claim 23, wherein said processing using artificial intelligence includes comparing the collected image data to a machine learning corpus derived at least in part from image data of different users.

27. The method of claim 23, wherein the evaluation is further based upon past image data associated with the user's gait.

28. The method of claim 23, wherein the evaluation comprises a detection that the user has fallen.

29. The method of claim 23, wherein the remote server is at least partly embodied in a cloud-based machine learning platform.
